(19) United States
(12) Patent Application Publication
(10) Pub. No.: US 2022/0023166 A1
(43) Pub. Date: Jan. 27, 2022

(54) TANNING COMPOSITION

(71) Applicants: MARLEY NICOLE KNOX, SANTA MONICA, CA (US); FREDDI DANIEL KNOX, SANTA MONICA, CA (US)

(72) Inventors: MARLEY NICOLE KNOX, SANTA MONICA, CA (US); FREDDI DANIEL KNOX, SANTA MONICA, CA (US)

(21) Appl. No.: 16/937,594

(22) Filed: Jul. 24, 2020

(51) Int. Cl. A61K 8/19; A61Q 17/04

(52) U.S. Cl. CPC A61K 8/19; A61K 2800/43; A61K 2800/413; A61Q 17/04

(57) ABSTRACT

The present invention employs the use of nano carbon black particles to a tanning composition. The tanning composition so produced will have all the solar spectrum absorbing qualities as the black particles used.

### SUMMARY OF THE INVENTION

The present invention is a skin tanning composition comprising of carbon black particles to a cosmetic base. It is an object of the invention to provide a skin tanning composition comprising of carbon black particles to a cosmetic base, to absorb solar spectrum.

It is another objective of this present invention to provide carbon black particles to a skin tanning composition product, such as lotions, creams, oils, gels, balms, solutions, emulsions, foam, mousse, sprays and any other combinations thereof.

It is yet another objective to provide a skin tanning composition that provides more solar absorption than other skin tanning compositions.

The foregoing and other objects, features and advantages of the present invention will become more apparent with reference to the detailed description and accompanying drawing.

### BACKGROUND OF THE INVENTION

Carbon black is almost a pure elemental carbon in the form of colloidal particles that are produced by incomplete combustion or thermal decomposition of gaseous or liquid hydrocarbons under controlled conditions. Carbon black consists mainly of carbon and carbon black is the name of a common black pigment, traditionally produced from charred bone or wood or other material and has been used throughout history (see, “What Is Carbon Black”, ICBA International Carbon Black Association). Carbon black has excellent hiding power and absorbs light (UV) well (see, Donnet, Jean-Baptiste Donnet. Carbon Black: Science and Technology, Second Edition. Routlege.1993).

Tanning and/or suntan is a browning or brown color of the skin resulting from exposure to sunlight (see, “Suntan”, Dictionary. Internet. 2019). The Sun's rays contain two types of ultraviolet radiation that reach your skin (the epidermis): UVA and UVB. UVA radiation is what makes people tan. UVA rays penetrate to the lower layers of the epidermis, where they trigger cells called melanocytes to produce melanin. Melanin is the brown pigment that causes an increase in the epidermis, causing tanning and/or suntan (see “Tanning”, Patrice Hyde, M D, The Nemours Foundation).

The term Albedo refers to the amount of solar energy that gets reflected off of the Earth and lands back into space, thus the Albedo is part of the energy from sunlight that casts back into the atmosphere. Albedo helps us to know how well a surface reflects solar energy. Surfaces differ in absorbent ability but will always be in the range of between 0-1. Value “0”-If a score of zero is given, the conclusion is that the surface is highly receptive to light, meaning that the surface takes in all the light that comes in contact with it. It is characterized by black surfaces. Value “1”-This score is evidence that the surface does not absorb incoming light. It is characterized by white surfaces (see, “What is Albedo?”, World Atlas).

It is an object of the present invention to provide a skin tanning composition that employs the use of nano carbon black particles to a cosmetic base.

### BRIEF DESCRIPTION OF THE DRAWING

FIG. 1 is a schematic diagram illustrating carbon black particle of the present invention.

FIG. 2 is a diagram of the epidermis layer, which involved in skin tanning.

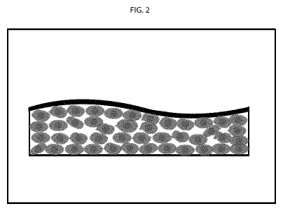

### DETAILED DESCRIPTION OF THE INVENTION

Hereinafter, embodiments of the present invention are described with reference to the drawings.

The present invention is in the technical field of carbon black particles and its integration to a skin tanning composition. Specifically, the present invention is in the technical field of the solar spectrum absorbing qualities of nano-carbon particles and its integration to a tanning composition. Based on the features of the invention, this is a skin tanning composition, which provides more solar spectrum absorption than other skin tanning compositions.

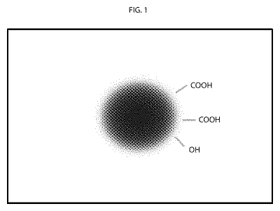

The drawings illustrates the carbon black particles used to influence the present invention's ability to absorb the highest level of solar spectrum to a cosmetic base. Referring to FIG. 1, shows the structure comprised of carbon black to a skin tanning composition to absorb solar spectrum. The embodiment illustrates various diagrams of a skin tanning composition that provides solar spectrum absorbing of carbon black particles to a cosmetic base; such as but not limited to creams, oils, gels, balms, solutions, emulsions, foam, mousse, sprays and any other combinations thereof.

In one embodiment, the present invention provides carbon black dyes to a cosmetic base to enhance UV radiation, providing a skin tanning composition, which absorbs full spectrum. Carbon black particles reflects very little lights in the visible spectrum by the material's Albedo, with the Albedo or reflectivity near zero (see, “The Energy Budget”, UCAR Center for Science Education). Black is the darkest color and absorbs the most heat. A black object absorbs all wavelengths of light and reflects none In the embodiment, said carbon black particles influences the ability for full solar spectrum absorption (see, “Which Colors Absorbs the Most Heat?”, University California Santa Barbara.

While the embodiments of the present invention has been described. It should be understood that various changes, adaptation and modification maybe made thereto. It should be understood, therefore, that the present invention is not limited to the details of the illustrated invention examples.

What is claimed is:

1. A skin tanning composition comprising:
nano carbon black particles to a cosmetic base.

2. A skin tanning composition according to claim 1, wherein said skin tanning composition and nano carbon black particles absorb solar spectrum.
